(19) United States
(12) Patent Application Publication
(10) Pub. No.: US 2021/0315501 A1
(43) Pub. Date: Oct. 14, 2021

(54) METHOD AND SYSTEM FOR DETECTING SPIRAL PATTERNS IN CANCELLATION TESTS

(71) Applicant: KONINKLIJKE PHILIPS N.V., Eindhoven (NL)

(72) Inventor: Jan Johannes Gerardus DE VRIES, Leende (NL)

(21) Appl. No.: 17/146,557

(22) Filed: Jan. 12, 2021

Related U.S. Application Data
(60) Provisional application No. 63/006,766, filed on Apr. 8, 2020.

(51) Int. Cl. A61B 5/16; G06F 3/041; G16H 10/20; G16H 50/20

(52) U.S. Cl. CPC A61B 5/168; G16H 50/20; G16H 10/20; G06F 3/0416

(57) ABSTRACT

A system configured to analyzing an individual's strategic cancellation test identification plan, comprising: a cancellation test pattern with a plurality of different symbols; a target symbol; a processor configured to: (i) receive an identification of a plurality of symbols within the visual cancellation test pattern; (ii) receive location information for each identified symbol; (iii) convert the location information to a polar coordinate; (iv) adjust by a first factor the polar coordinate of each identified symbol corresponding to a clockwise rotation, and adjust by a second factor the polar coordinate of each identified symbol corresponding to a counterclockwise rotation; and (v) analyze the adjusted polar coordinates to determine the strategic cancellation test identification plan; and a user interface configured to provide a report comprising the determined strategic cancellation test identification plan.

### FIELD OF THE DISCLOSURE

The present disclosure is directed generally to methods and systems for detecting a spiral cancellation pattern utilized by a patient in a visual cancellation test.

### BACKGROUND

A cancellation test is a tool utilized in clinical and research settings to measure an individual's attention/concentration, and to diagnose or analyze visual neglect resulting from vision and/or brain conditions. The cancellation test consists of a visual display—electronic or paper—with a pattern of letters, numbers, or symbols. Examples of cancellation tests include letter, star, o-search, and many other options. The pattern may be highly structured or more randomized. The individual is instructed to cross out, circle, or otherwise identify a particular target which is interspersed within the pattern. The frequency of the target within the pattern can be raised or lowered to make the test less difficult or more challenging, respectively.

The individual's progress is monitored in terms of time spent on the cancellation test, and can be scored based on how many of the target are successfully identified and/or how many non-target items are incorrectly identified within the pattern. The timing and score, as well as the pattern of identified or misidentified items, can be used to quantify attention or concentration, to quantify or diagnose visual neglect, motor issues, and more. For example, slower responses and more errors can indicate an underlying issue such as physical motor damage, brain conditions, or visual neglect.

In addition to measuring attention/concentration and visual neglect, a cancellation test can be utilized to measure the individual's cancellation strategy. Given that the patients are not instructed to use a particular search strategy, they will use a strategy that they consider optimal pursuant to their mental abilities. For example, it is known that stroke patients with varying brain damage, including based on the damaged region and size, exhibit different search pattern strategies. Thus, it is vital to be able to detect a structured search strategy versus a more random search strategy.

Currently, the outcome of a cancellation test is an ordered series of (xᵢ, yⱼ) coordinate pairs indicating the position of each identified item from the pattern indexed (order wise) by i∈{1, 2, . . . , n}. Typically, left-right and up-down directions are tracked during the cancellation test, which can measure left-right and up-down search strategies used by the individual. However, current cancellation test methodologies cannot track or detect spiraling or circular search strategies used by the individual, which can result in that type of search strategy being incorrectly classified as a deficit or other issue.

### SUMMARY OF THE DISCLOSURE

There is a continued need for methods and systems that more accurately analyze an individual's strategic search pattern during a cancellation test by enabling detection and analysis of spiraling and circular search strategies. The present disclosure is directed to inventive methods and systems for analyzing a person's strategic identification plan during a cancellation test. Various embodiments and implementations herein are directed to a system and method that displays a visual cancellation test to an individual. The cancellation test includes a plurality of different symbols each associated with a coordinate indicating a location of that symbol. The patient is given an indication of a target symbol in the cancellation test pattern to identify by circling, crossing out, or other method. The system receives from the patient an identification of a plurality of symbols within the visual cancellation test pattern, with the goal being the identification of the target symbol. The system determines or receives the location of each patient-identified symbol within the pattern, and converts that location information to of each of the plurality of identified symbols to polar coordinates. Based on the respective polar coordinates, each of the patient-identified symbols is analyzed to determine whether it corresponded to a clockwise or counterclockwise rotation during the patient's identification. The system adjusts the polar coordinate of each identified symbol corresponding to a clockwise rotation by a first factor, and adjusts the polar coordinate of each identified symbol corresponding to a counterclockwise rotation by a second factor. The system analyzes the resulting adjusted polar coordinates to determine the strategic identification plan carried out by the patient during the identification of the plurality of symbols within the visual cancellation test pattern. The system can then generate a report comprising information about the spiraling or circular search strategy utilized by the patient during the test.

Generally, in one aspect, is a system for analyzing an individual's strategic cancellation test identification plan. The system includes: (i) a cancellation test pattern, comprising a plurality of each of a plurality of different symbols, each symbol associated with location information for the symbol within the pattern; (ii) a target symbol, wherein the target symbol is at least one of the plurality of different symbols in the cancellation test pattern; (iii) a processor configured to: receive an identification of a plurality of symbols within the visual cancellation test pattern; receive, for each of the plurality of identified symbols, the associated location information; convert the received location information for each of the plurality of identified symbols to a polar coordinate; adjust by a first factor the polar coordinate of each identified symbol corresponding to a clockwise rotation, and adjust by a second factor the polar coordinate of each identified symbol corresponding to a counterclockwise rotation; and analyze the resulting adjusted polar coordinates to determine the strategic cancellation test identification plan carried out by the patient during the identification of the plurality of symbols; and (iv) a user interface configured to provide a report comprising the determined strategic cancellation test identification plan carried out by the patient during the identification of the plurality of symbols.

According to an embodiment, the processor is configured to convert the associated location information of each of the plurality of identified symbols to a polar coordinate using a function that maps Cartesian coordinates to polar coordinates.

According to an embodiment, the processor is configured to, when adjusting the polar coordinates using the first factor or second factor, determine a first derivative comprising a difference between each identified symbol compared to its predecessor.

According to an embodiment, the processor is configured to adjust a polar coordinate by a first factor when the first derivative exceeds π. According to an embodiment, adjusting by the first factor comprises subtracting 2π from the polar coordinate.

According to an embodiment, the processor is configured to adjust a polar coordinate by a second factor when the first derivative exceeds −π. According to an embodiment, adjusting by the second factor comprises adding 2π to the polar coordinate.

According to an embodiment, the processor is configured to, when analyzing the resulting adjusted polar coordinates, compare the adjusted polar coordinates to unadjusted polar coordinates.

According to an embodiment, the processor is further configured to determine a standard deviation of the adjusted polar coordinates when comparing against the unadjusted polar coordinates.

According to an embodiment, the processor is alternatively configured to adjust by a first factor the polar coordinate of a reference signal corresponding to each identified symbol corresponding to a clockwise rotation, and adjust by a second factor the polar coordinate of the reference signal corresponding to each identified symbol corresponding to a counterclockwise rotation, to generate adjusted polar coordinates for the reference signal; and the processor is further alternatively configured to analyze the generated adjusted polar coordinates for the reference signal to determine the strategic cancellation test identification plan carried out by the patient during the identification of the plurality of symbols.

According to another aspect is a method for analyzing an individual's strategic cancellation test identification plan. The method includes: (i) providing, to the individual, a visual cancellation test pattern comprising a plurality of each of a plurality of different symbols, wherein each symbol is associated with location information for that symbol; (ii) providing, to the individual, an indication of a target symbol in the cancellation test pattern to identify, wherein the target symbol is one of the plurality of different symbols; (iii) receiving, from the individual, an identification of a plurality of symbols within the visual cancellation test pattern; (iv) receiving, for each of the plurality of identified symbols, the associated location information; (v) converting the received location information for each of the plurality of identified symbols to a polar coordinate; (vi) adjusting, by a first factor, the polar coordinate of each identified symbol corresponding to a clockwise rotation and adjusting, by a second factor, the polar coordinate of each identified symbol corresponding to a counterclockwise rotation; (vii) analyzing the resulting adjusted polar coordinates to determine the strategic cancellation test identification plan carried out by the patient during the identification of the plurality of symbols.

According to an embodiment, the method further includes the step of generating a report comprising the determined strategic cancellation test identification plan carried out by the patient during the identification of the plurality of symbols.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1A is a flowchart of a method for analyzing a person's strategic identification plan during a cancellation test, in accordance with an embodiment.

FIG. 1B a flowchart of a method for analyzing a person's strategic identification plan during a cancellation test, in accordance with an embodiment.

FIG. 2A is a series of graphs depicting a clockwise spiral pattern used by an individual during a cancellation test, in accordance with an embodiment.

FIG. 2B is a series of graphs depicting a counterclockwise spiral pattern used by an individual during a cancellation test, in accordance with an embodiment.

FIG. 2C is a series of graphs depicting a left-to-right pattern used by an individual during a cancellation test, in accordance with an embodiment.

FIG. 2D is a series of graphs depicting a random pattern used by an individual during a cancellation test, in accordance with an embodiment.

FIG. 3 is a schematic representation of a system for analyzing a person's strategic identification plan during a cancellation test, in accordance with an embodiment.

### DETAILED DESCRIPTION OF EMBODIMENTS

The present disclosure describes various embodiments of a system and method that analyzes an individual's strategic search pattern during a cancellation test to enable detection and analysis of spiraling and circular search strategies. More generally, Applicant has recognized and appreciated that it would be beneficial to provide a method that more accurately analyzes a person's strategic identification plan during a cancellation test. To improve the identification of a strategic identification plan, the system displays a visual cancellation test to an individual comprising a plurality of different symbols each associated with a coordinate indicating a location of that symbol. The individual is told which target symbol in the cancellation test pattern to identify using a particular identification method. The system receives from the patient an identification of a plurality of symbols within the visual cancellation test pattern, with the goal being the identification of the target symbol. The system receives the location of each patient-identified symbol, and converts the location of each of the plurality of identified symbols to a polar coordinate. Based on the respective polar coordinates, each of the patient-identified symbols is analyzed to determine whether it corresponded to a clockwise or counterclockwise rotation during the patient's identification. The system adjusts the polar coordinate of each identified symbol corresponding to a clockwise rotation by a first factor, and adjusts the polar coordinate of each identified symbol corresponding to a counterclockwise rotation by a second factor. The system analyzes the resulting adjusted polar coordinates to determine the strategic identification plan carried out by the patient during the identification of the plurality of symbols within the visual cancellation test pattern. The system can then generate a report comprising information about the spiraling or circular search strategy utilized by the patient during the test.

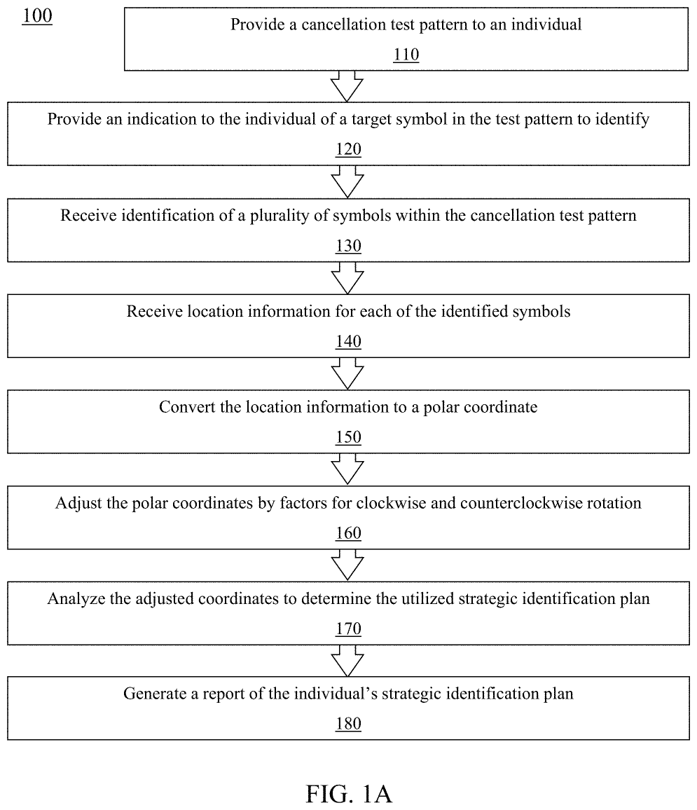

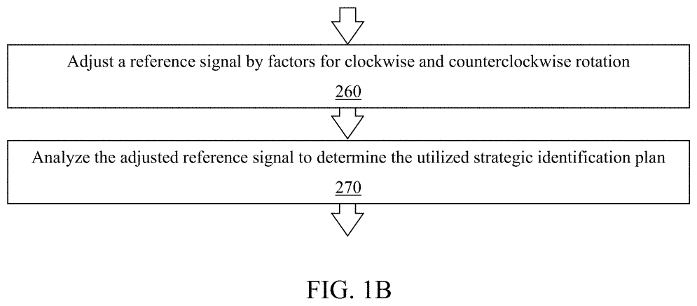

Referring to FIG. 1A, in one embodiment, is a flowchart of a method 100 for analyzing a person's strategic identification plan during a cancellation test using a cancellation test system. The cancellation test system may be any of the systems described or otherwise envisioned herein, and may comprise any of the components described or otherwise envisioned herein.

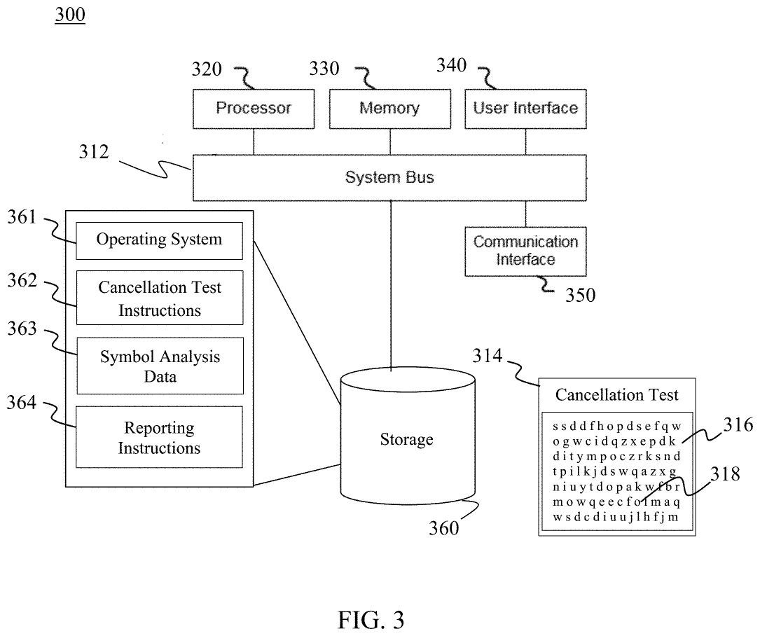

At step 110 of the method, a cancellation test pattern 314 (shown in FIG. 3) is provided to an individual. The individual can be anyone, such as for example someone for whom a cancellation test is warranted or otherwise indicated to analyze or diagnose a potential issue. Cancellation tests can be utilized for a wide variety of potential conditions, including but not limited to an individual's attention/concentration, visual neglect resulting from vision and/or brain conditions, and strategic planning among many others. Thus, an individual that may potentially be affected by a condition or have one of these issues may be asked to participate in a cancellation test in order to provide information that may facilitate analysis and/or diagnosis. The individual may participate in the cancellation test at a medical or other care facility, at a medical professional's office, at home, or at any other location.

The cancellation test may be provided to the individual electronically or physically such as on paper. An electronic cancellation test may be displayed on a monitor, touchscreen, or any other method of electronic display. The cancellation test comprises a pattern with a plurality of different symbols 316 (shown in FIG. 3), and typically with a plurality of each type of symbol. A symbol can be anything that can be formed into a pattern. For example, the symbols may be letters, numbers, shapes, and/or any other displayable or printable item. The symbols form a pattern on the paper or display, and the pattern may be highly structured or more randomized. Typically, a single one of the plurality of different symbols is the target symbol, which is the target of the individual's attention during the test. For example, if the cancellation test comprises a plurality of different letters such as all the letters of the alphabet distributed throughout the page or screen, the target may be the letter ‘o’ such that the individual is instructed to identify all the instances of the letter ‘o’ on the page or screen. In some cancellation tests, two or more symbols are targets and they may operate independently or in tandem such as in pairs or other spatial relationship.

Providing the cancellation test to the individual can be as simple as handing the individual a printout of the test, handing them a portable electronic device with a displayed test, displaying the test on a monitor or other screen or type of display, or otherwise giving the person the cancellation test.

According to an embodiment, each symbol in the pattern is associated with location information that indicates the physical location of that symbol. The location and coordinate system may be with regard to a particular point of the pattern, such as a point of origin. The point may be a corner, the center, or any other point. Associated can mean simply that a symbol possesses, by virtue of being included in the pattern on paper or on the display, a coordinate relative to any point of the test and preferably relative to a particular point utilized for calculating coordinates. Associated may alternatively mean that the system knows the coordinate of each symbol in the pattern and has associated that coordinate with the symbol in memory, a data table, or other association mechanism. According to an embodiment, the location information comprises Cartesian coordinates. Thus, each symbol may be associated in memory or some other data structure with Cartesian coordinates for that symbol. Alternatively, the system may be configured to determine the Cartesian coordinates for a symbol, including before, during, or after selection of that symbol by the individual.

At step 120 of the method, an identification of a target symbol 318 (shown in FIG. 3) in the cancellation test pattern is provided to the individual. As described herein, the target symbol is one or more of the plurality of different symbols in the pattern provided to the individual. As an initial step of the test, the individual is provided with an indication or identification of which of the plurality of different symbols in the pattern is the target symbol. The individual will identify, using any of a wide variety of different possible methods, as many of the target symbols in the pattern as possible.

An indication or identification of the target symbol can be given to the individual using any method. It can be a verbal instruction provided to the individual locally or remotely. Additionally or alternatively, the indication or identification of the target symbol can be given to the individual by visibly displaying the target symbol, such as on a physical copy of the test or on a monitor, screen, or other display. For example, the target symbol can be printed on one portion of a physical copy of the test separate from the pattern, or the target symbol can be displayed on the monitor, screen, or other display separate from the pattern.

Providing the indication or identification of the target symbol can be accompanied with an instruction to the individual, such as an instruction to identify as many instances of the target symbol as possible, without incorrectly identifying non-target symbols. Many different instructions are possible. Timing information may also be provided to the individual, such as informing the individual that the test will be timed and/or that there is a certain amount of time within which to complete the task.

At step 130 of the method, the cancellation test system receives from the individual an identification of a plurality of symbols within the cancellation test pattern. According to an embodiment, the system monitors the individual's interactions with the test. Monitoring the interaction could be receiving input from a touchscreen, receiving video input of the individual's interaction with the test, or any other method of identifying when and which symbol(s) are selected or otherwise identified by the individual. The identification can be received in real-time or after the test. If the test is a physical pen-and-paper test, the system may receive input about which symbols were selected after the test, such by scanning the marked-up test or otherwise receiving information about which symbols were received and preferably in what order.

Identification of the target symbol by the individual can be accomplished via any method for creating or providing input. For example, the individual can circle each instance of the target symbol, can cross-out or ‘x-out’ each instance of the target symbol, can touch each instance of the target symbol with a finger, or can use any other method for identifying the target symbol.

Current state of the art systems measure an individual's strategic search pattern using left/right and up/down tracking. The system correlates x coordinates (left/right) or y coordinates (up/down) with their index (i.e., correlate X={xᵢ} with corresponding I={i}, or analogously for Y={yⱼ}). This correlation will be high—close to 1—if the individual exhibits a global strategy of crossing out items from left to right. The correlation will be low—close to −1—if the individual exhibits a global strategy of crossing out items from right to left. The correlation will be close to 0 when a more random search is performed, and analogously for top/bottom, and are thus good indicators for determining whether a linear search strategy was used as compared to a more random search strategy. However, some individuals may exhibit a highly structured search strategy in the form of a spiral or circular pattern. Unfortunately these patterns will show close-to-zero values for the abovementioned correlations in the x and y direction, and would thus incorrectly be categorized as non-structured or random search pattern. Accordingly, there is a need to detect and quantify spiral/circular patterns in cancellation tests, and thus allow for a better quantification of the level of strategic planning in patients.

At step 140 of the method, the cancellation test system determines and/or receives, for each of the plurality of symbols identified by the individual, a location of the symbol. According to an embodiment, the system knows the coordinate of each symbol in the pattern and has associated that coordinate with the symbol in memory, a data table, or other association mechanism. The system can therefore receive or retrieve the location information for a symbol, such as when the symbol is selected. According to another embodiment, the system determines or calculates location information for a symbol, such as when the symbol is selected, and receives that calculated or determined location information. According to an embodiment, the location information comprises Cartesian coordinates. Thus, each symbol may be associated in memory or some other data structure with Cartesian coordinates for that symbol. Alternatively, the system may be configured to determine the Cartesian coordinates for a symbol, including before, during, or after selection of that symbol by the individual.

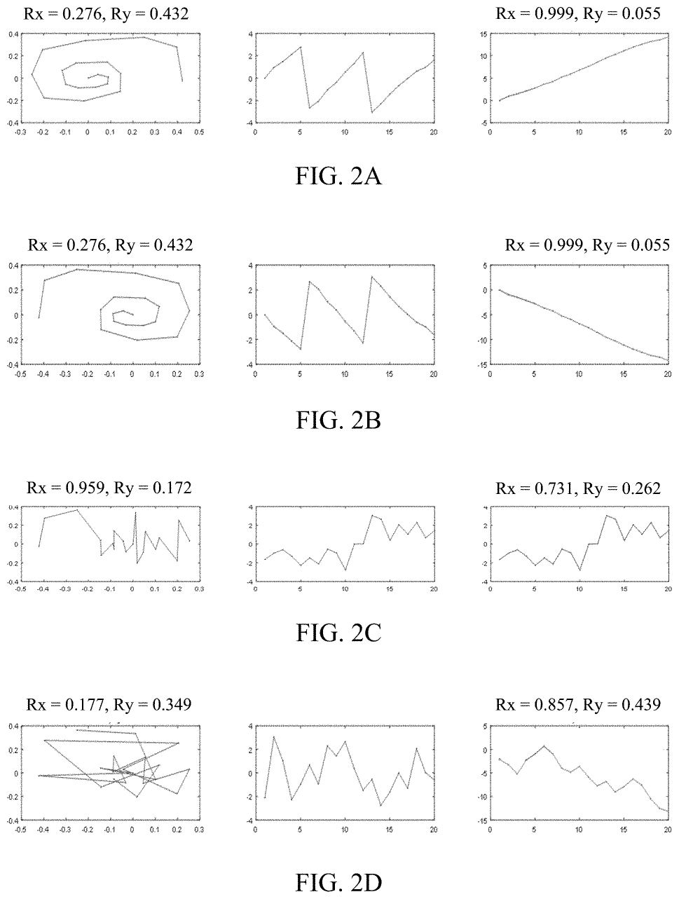

Referring to FIGS. 2A-2D are a series of graphs showing connected dots of positions in the order that the corresponding symbols have been identified by an individual in each of four different cancellation tests. FIG. 2A shows a clockwise spiral pattern used by the individual, FIG. 2B shows a counterclockwise spiral pattern used by the individual, FIG. 2C shows a left-to-right or increasing x-coordinate pattern used by the individual, and FIG. 2D shows a completely random pattern used by the individual.

According to an embodiment, the point of origin of the graphs where x=0 and y=0 is the center of the anticipated spiral and thus chosen as the center of the symbol pattern. Above each of the leftmost panel in each figure is the Rx and Ry value calculated using the outdated prior art methods for determining strategic planning. As expected, the Rx value for the left-right pattern in FIG. 2C is very close to 1 (0.959). In contrast, the Rx and Ry values of the spiral patterns in FIG. 2A (Rx=0.276, Ry=0.432) and FIG. 2B (Rx=0.276, Ry=0.432) are very similar to the random pattern in FIG. 2D (Rx=0.177, Ry=0.349). This is due to the inability of prior art systems to determine spiral patterns.

At step 150 of the method, the cancellation test system converts the received location information for each of the plurality of identified symbols to a polar coordinate. According to an embodiment, the cancellation test system obtains the (x,y) Cartesian coordinates of each identified symbol and calculates their polar coordinate. Any method can be utilized to convert the information to polar coordinates. According to an embodiment, the conversion is performed by a function that maps Cartesian coordinates to polar coordinates. As just one example, an arctan 2 function can be utilized to calculate their polar coordinate from the (x,y) coordinates. Referring to FIGS. 2A-2D, the middle panel of each figure is the plot of the calculated polar coordinates. According to an embodiment, the polar coordinate signal can be called {αᵢ}. As shown in FIGS. 2A and 2B, a spiral pattern shows a sawtooth pattern in their coordinates, while the linear (FIG. 2C) and random (FIG. 2D) pattern show a much more random coordinate pattern. The sawtooth pattern actually wraps around the values of ±π.

Once the polar coordinates for each of the symbols identified by the individual are calculated, the system can save or store this information in a memory for future use. Alternatively and/or additionally, the system can use this information such as plotting graphs as shown in FIGS. 2A-2D.

At step 160 of the method, the cancellation test system adjusts, by a first factor, the polar coordinate of each identified symbol corresponding to a clockwise rotation, or adjusts, by a second factor, the polar coordinate of each identified symbol corresponding to a counterclockwise rotation. A final signal is determined by the adjusted coordinates.

The first factor and second factor can be a variety of possible factors. According to an embodiment, the system identifies the first derivative of the signal. That signal can be called Δα. When Δα exceeds π, there is an upward jump that corresponds to the individual having fulfilled a counterclockwise rotation. To compensate for that, the system finds all i for which Δαᵢ>π, and subtracts 2π from that sample and from all the samples following (i.e., all samples αⱼ with j∈{i, . . . , n}. Thus, the polar coordinate of each identified symbol corresponding to a counterclockwise rotation is adjusted by this factor. Similarly, when Δα exceeds −π, there is a downward jump that corresponds to the individual having fulfilled a clockwise rotation. To compensate for that, the system adds 2π to those samples and their successors. Thus, the polar coordinate of each identified symbol corresponding to a clockwise rotation is adjusted by this factor. The resulting signal, following the adjustments, can be called {\tilde{α}ᵢ}.

According to another embodiment, the first factor and/or second factor comprise degrees. When angles for the polar coordinates are measured in degrees, the first and second factors can be degrees. For example, rather than adjusting by π or 2π, the system adjusts by 180 degrees or 360 degrees, respectively. Many other factors and adjustment methods are possible.

Once the resulting signal {\tilde{α}ᵢ} is calculated, the system can save or store this information in a memory for future use. Alternatively and/or additionally, the system can use this information such as plotting graphs as shown in FIGS. 2A-2D.

At step 170 of the method, the cancellation test system analyzes the resulting adjusted polar coordinates to determine the strategic identification plan carried out by the patient during the identification of the plurality of symbols within the visual cancellation test pattern. There are a wide variety of methods for analyzing the resulting signal {\tilde{α}ᵢ}. According to an embodiment, the resulting line can be used again to determine how well it correlates with the indices of the samples, similar to the state-of-art technique that looks the x or y coordinates.

According to another embodiment, the system can determine the standard deviation of the residuals when comparing against the linear regressor of the signal. The system can calculate the linear regression line through the points {(i, \tilde{α}ᵢ)}, such as taking the index as the first coordinate and the corrected angle as the second coordinate, determining per sample how much it differs from the regressor-value at that point (the ‘residual value’), and then determining the standard deviation of these differences. This provides an indication of whether the individual utilized a spiral or circular pattern during the test.

Referring to FIGS. 2A and 2B, for example, the Rx of the initial leftmost panel is most similar to a random pattern, while the adjusted Rx of the rightmost panel with the post-processed signal is nearly 1, indicating a spiral or circular pattern used during the test. For example, the Rx value for the counterclockwise pattern utilized in FIG. 2A is initially 0.276, but it is adjusted to 0.999 using the methods and system described herein. Similarly, the Rx value for the clockwise pattern utilized in FIG. 2A is initially 0.276, but it is adjusted to 0.999 using the methods and system described herein. Conversely, the Rx value for the linear pattern utilized in FIG. 2C is initially 0.959, but is adjusted to 0.731 using the methods and system described herein, indicating that a spiral or circular pattern was not used during the test. Similarly, the resulting value for the random pattern in FIG. 2D is 0.857, again indicating that a spiral or circular pattern was not used during the test.

The system can perform the analysis and calculate the final signal using any of a wide variety of methods as described or otherwise envisioned herein. According to just one embodiment, a software package such as MATLAB® can be utilized to perform one or more steps of the conversion. For example, the following MATLAB code can be utilized, although it is provided as just one non-limiting example:

% calculate angle (polar coordinate)

angle=a tan 2(x2,y2);

angle2=angle+cumsum([0;−2*pi.*(diff(angle)>pi)]) . . . +cumsum([0;+2*pi.*(diff(angle)<−pi)]);

X=[ones(N,1),(1:N)′];

Y=angle2;

% suppose Y=b*X(+epsilon)

[b,bint,res]=regress(Y(:,1),X);

r2=std(res)/sqrt(N); % standard error w.r.t. regression line

r=corr((1:N)′,angle2); % calculate the traditional measure

Referring to FIG. 1B, in one embodiment, is a flowchart of a method 200 for analyzing a person's strategic identification plan during a cancellation test using a cancellation test system. The cancellation test system may be any of the systems described or otherwise envisioned herein, and may comprise any of the components described or otherwise envisioned herein. Method 200 is similar to method 100, but at step 260 the system instead adjusts by a first factor the polar coordinate of a reference signal corresponding to each identified symbol corresponding to a clockwise rotation. Thus, the system is adjusting a reference signal at a location equivalent to the identified symbol location. The system also adjusts by a second factor the polar coordinate of the reference signal corresponding to each identified symbol corresponding to a counterclockwise rotation, to generate adjusted polar coordinates for the reference signal. At 270, the system instead analyzes the generated adjusted polar coordinates for the reference signal to determine the strategic cancellation test identification plan carried out by the patient during the identification of the plurality of symbols. The method then returns to method 100 in FIG. 1A.

At step 180 of method 100 in FIG. 1A, the cancellation test system generates and provides a report comprising the results of the analysis. The report may comprise any of the data or information generated or obtained as described or otherwise envisioned herein. The report may be electronic or printed, and may be stored. For example, the report may comprise a graph, a text-based file, or any other format. The report may be configured to allow easy analysis and extraction of information.

According to an embodiment, the cancellation test system may visually display the results of the analysis, such as the final Rx and/or Ry coordinate information, and/or a graph comprising the information, on a screen or other display method. According to an embodiment, the report or information may be stored in temporary and/or long-term memory or other storage. Additionally and/or alternatively, the report or information may be communicated or otherwise transmitted to another system, recipient, process, device, and/or other local or remote location.

According to an embodiment, once the report or information is generated, it can be provided to a clinician, researcher, or other user to review. The recipient may utilize the report to determine whether the individual utilized a spiral or circular pattern during the test, or perhaps utilized another type of pattern. According to an embodiment, a clinician may, for example, diagnose a disorder or hypothesize the existence of a particular disorder based on the output of the report. The clinician may additional or alternatively select a specific treatment based on the output of the report.

Referring to FIG. 3, in one embodiment, is a schematic representation of a cancellation test system 300 configured to analyze a person's strategic identification plan during a cancellation test. The cancellation test system 300 may be any of the systems described or otherwise envisioned herein, and may comprise any of the components described or otherwise envisioned herein.

According to an embodiment, system 300 comprises one or more of a processor 320, memory 330, user interface 340, communications interface 350, and storage 360, interconnected via one or more system buses 312. It will be understood that FIG. 3 constitutes, in some respects, an abstraction and that the actual organization of the components of the system 300 may be different and more complex than illustrated.

According to an embodiment, system 300 comprises a processor 320 capable of executing instructions stored in memory 330 or storage 360 or otherwise processing data to, for example, perform one or more steps of the method. Processor 320 may be formed of one or multiple modules. Processor 320 may take any suitable form, including but not limited to a microprocessor, microcontroller, multiple microcontrollers, circuitry, field programmable gate array (FPGA), application-specific integrated circuit (ASIC), a single processor, or plural processors.

Memory 330 can take any suitable form, including a non-volatile memory and/or RAM. The memory 330 may include various memories such as, for example L1, L2, or L3 cache or system memory. As such, the memory 330 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 300. It will be apparent that, in embodiments where the processor implements one or more of the functions described herein in hardware, the software described as corresponding to such functionality in other embodiments may be omitted.

User interface 340 may include one or more devices for enabling communication with a user. The user interface can be any device or system that allows information to be conveyed and/or received, and may include a display, a mouse, and/or a keyboard for receiving user commands. In some embodiments, user interface 340 may include a command line interface or graphical user interface that may be presented to a remote terminal via communication interface 350. The user interface may be located with one or more other components of the system, or may located remote from the system and in communication via a wired and/or wireless communications network.

Communication interface 350 may include one or more devices for enabling communication with other hardware devices. For example, communication interface 350 may include a network interface card (NIC) configured to communicate according to the Ethernet protocol. Additionally, communication interface 350 may implement a TCP/IP stack for communication according to the TCP/IP protocols. Various alternative or additional hardware or configurations for communication interface 350 will be apparent.

Storage 360 may include one or more machine-readable storage media such as read-only memory (ROM), random-access memory (RAM), magnetic disk storage media, optical storage media, flash-memory devices, or similar storage media. In various embodiments, storage 360 may store instructions for execution by processor 320 or data upon which processor 320 may operate. For example, storage 360 may store an operating system 361 for controlling various operations of system 300.

It will be apparent that various information described as stored in storage 360 may be additionally or alternatively stored in memory 330. In this respect, memory 330 may also be considered to constitute a storage device and storage 360 may be considered a memory. Various other arrangements will be apparent. Further, memory 330 and storage 360 may both be considered to be non-transitory machine-readable media. As used herein, the term non-transitory will be understood to exclude transitory signals but to include all forms of storage, including both volatile and non-volatile memories.

While system 300 is shown as including one of each described component, the various components may be duplicated in various embodiments. For example, processor 320 may include multiple microprocessors that are configured to independently execute the methods described herein or are configured to perform steps or subroutines of the methods described herein such that the multiple processors cooperate to achieve the functionality described herein. Further, where one or more components of system 300 is implemented in a cloud computing system, the various hardware components may belong to separate physical systems. For example, processor 320 may include a first processor in a first server and a second processor in a second server. Many other variations and configurations are possible.

According to an embodiment, storage 360 of cancellation test system 300 may store one or more algorithms and/or instructions to carry out one or more functions or steps of the methods described or otherwise envisioned herein. For example, processor 320 may comprise cancellation test instructions 362, symbol analysis instructions 363, and/or reporting instructions 364.

According to an embodiment, cancellation test instructions 362 direct the system to conduct the cancellation test. For example, the instructions comprise information for displaying or otherwise providing the visual cancellation test pattern to the individual via a display or via a printout. The instructions may create the pattern pursuant to one or more parameters, or may utilize a pre-existing pattern. The system may provide the cancellation test to the individual using any of the methods or approaches described or otherwise envisioned herein. The system also provides an indication to the individual of which of the plurality of symbols in the pattern is the target symbol(s).

The instructions further enable the system to receive input from the user during or after the cancellation test. Identification of the target symbol by the individual can be accomplished via any method for creating or providing input. According to an embodiment, the system monitors the individual's interactions with the test, which could include receiving input from a touchscreen, receiving video input of the individual's interaction with the test, or any other method of identifying when and which symbol(s) are selected or otherwise identified by the individual. If the test is a physical pen-and-paper test, the system may receive input about which symbols were selected after the test, such by scanning the marked-up test or otherwise receiving information about which symbols were received and preferably in what order.

According to an embodiment, symbol analysis instructions 363 direct the system to process the input received from the individual regarding selecting symbols during the cancellation test. The symbol analysis instructions direct the system to determine and/or receive, for each of the plurality of symbols identified by the individual, location information for each identified symbol.

The symbol analysis instructions also direct the system to convert the location information for each of the plurality of identified symbols to a polar coordinate. According to an embodiment, the cancellation test system obtains the (x,y) coordinates of each identified symbol and calculates the respective polar coordinate. Any method can be utilized to convert the information to polar coordinates. As just one example, an arctan 2 function can be utilized to calculate their polar coordinate from the (x,y) coordinates.

The symbol analysis instructions also direct the system to adjust, by a first factor, the polar coordinate of each identified symbol corresponding to a clockwise rotation, or adjust, by a second factor, the polar coordinate of each identified symbol corresponding to a counterclockwise rotation. A final signal is then determined by the adjusted coordinates.

For example, according to one embodiment, the system identifies the first derivative of the signal (Δα), which is the difference between each identified symbol compared to its predecessor. When Δα exceeds π, there is an upward jump that corresponds to the individual having fulfilled a counterclockwise rotation. To compensate for that, the system finds all i for which Δαᵢ>π, and subtracts 2π from that sample and from all the samples following. Thus, the polar coordinate of each identified symbol corresponding to a counterclockwise rotation is adjusted by this factor. Similarly, when Δα exceeds −π, there is a downward jump that corresponds to the individual having fulfilled a clockwise rotation. To compensate for that, the system adds 2π to those samples and their successors. Thus, the polar coordinate of each identified symbol corresponding to a clockwise rotation is adjusted by this factor. The resulting signal is {\tilde{α}}.

The symbol analysis instructions also direct the system to analyze the resulting signal {\tilde{α}ᵢ} to determine the strategic identification plan carried out by the patient during the identification of the plurality of symbols within the visual cancellation test pattern. There are a wide variety of methods for analyzing the resulting signal {\tilde{α}ᵢ}. According to an embodiment, the resulting line can be used again to determine how well it correlates with the indices of the samples, similar to the state-of-art technique that looks the x or y coordinates. According to another embodiment, the system can determine the standard deviation of the residuals when comparing against the linear regressor of the signal. The system can calculate the linear regression line through the points {(i, \tilde{α}ᵢ)}, such as taking the index as the first coordinate and the corrected angle as the second coordinate, determining per sample how much it differs from the regressor-value at that point (the ‘residual value’), and then determining the standard deviation of these differences. This provides an indication of whether the individual utilized a spiral or circular pattern during the test.

According to an embodiment, reporting instructions 364 direct the system to generate and provide a report comprising the results of the analysis. The report may comprise any of the data or information generated or obtained as described or otherwise envisioned herein. The report may be electronic or printed, and may be stored. For example, the report may comprise a graph, a text-based file, or any other format. The report may be configured to allow easy analysis and extraction of information.

The reporting instructions 364 may direct the system to store the generated report or information in temporary and/or long-term memory or other storage. This may be local storage within system 300 or associated with system 300, or may be remote storage which received the report or information from or via system 300. Additionally and/or alternatively, the report or information may be communicated or otherwise transmitted to another system, recipient, process, device, and/or other local or remote location.

The reporting instructions 364 may direct the system to provide the generated report to a user or other system. For example, the cancellation test system may visually display information about the cancellation test results, the individual, the search strategy utilized by the individual, and/or any other information on the user interface, which may be a screen or other display.

According to an embodiment, the cancellation test system and approach described or otherwise envisioned herein enables a clinician, researcher, or other user to more accurately determine a circular or spiral strategic search utilized by an individual during a cancellation test. This significantly improves the analytical, diagnostic, and/or treatment decisions of the clinician, researcher, or other user. This is especially important for motor deficiencies, brain issues, and visual issues, for which cancellation testing is so commonly utilized.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A cancellation test system configured to analyzing an individual's strategic cancellation test identification plan, comprising:
a cancellation test pattern, comprising a plurality of each of a plurality of different symbols, each symbol associated with location information for the symbol within the pattern;
a target symbol, wherein the target symbol is at least one of the plurality of different symbols in the cancellation test pattern;
a processor configured to: (i) receive an identification of a plurality of symbols within the visual cancellation test pattern; (ii) receive, for each of the plurality of identified symbols, the associated location information; (iii) convert the received location information for each of the plurality of identified symbols to a polar coordinates; (iv) adjust by a first factor the polar coordinate of each identified symbol corresponding to a clockwise rotation, and adjust by a second factor the polar coordinate of each identified symbol corresponding to a counterclockwise rotation; and (v) analyze the resulting adjusted polar coordinates to determine the strategic cancellation test identification plan carried out by the patient during the identification of the plurality of symbols; and
a user interface configured to provide a report comprising the determined strategic cancellation test identification plan carried out by the patient during the identification of the plurality of symbols.

2. The system of claim 1, wherein the processor is configured to convert the associated location information of each of the plurality of identified symbols to a polar coordinate using a function that maps Cartesian coordinates to polar coordinates.

3. The system of claim 1, wherein the processor is configured to, when adjusting the polar coordinates using the first factor or second factor, determine a first derivative comprising a difference between each identified symbol compared to its predecessor.

4. The system of claim 3, wherein the processor is configured to adjust a polar coordinate by a first factor when the first derivative exceeds π.

5. The system of claim 4, wherein adjusting by the first factor comprises subtracting 2π from the polar coordinate.

6. The system of claim 3, wherein the processor is configured to adjust a polar coordinate by a second factor when the first derivative exceeds −π.

7. The system of claim 6, wherein adjusting by the second factor comprises adding 2π to the polar coordinate.

8. The system of claim 1, wherein the processor is configured to, when analyzing the resulting adjusted polar coordinates, compare the adjusted polar coordinates to unadjusted polar coordinates of a reference signal.

9. The system of claim 1, wherein at (iv) the processor is alternatively configured to adjust by a first factor the polar coordinate of a reference signal corresponding to each identified symbol corresponding to a clockwise rotation, and adjust by a second factor the polar coordinate of the reference signal corresponding to each identified symbol corresponding to a counterclockwise rotation, to generate adjusted polar coordinates for the reference signal; and
further wherein at (v) the processor is alternatively configured to analyze the generated adjusted polar coordinates for the reference signal to determine the strategic cancellation test identification plan carried out by the patient during the identification of the plurality of symbols.

10. A method for analyzing an individual's strategic cancellation test identification plan, comprising:
providing, to the individual, a visual cancellation test pattern comprising a plurality of each of a plurality of different symbols, wherein each symbol is associated with location information for that symbol;
providing, to the individual, an indication of a target symbol in the cancellation test pattern to identify, wherein the target symbol is one of the plurality of different symbols;
receiving, from the individual, an identification of a plurality of symbols within the visual cancellation test pattern;
receiving, for each of the plurality of identified symbols, the associated location information;
converting the received location information for each of the plurality of identified symbols to a polar coordinate;
adjusting, by a first factor, the polar coordinate of each identified symbol corresponding to a clockwise rotation and adjusting, by a second factor, the polar coordinate of each identified symbol corresponding to a counterclockwise rotation;
analyzing the resulting adjusted polar coordinates to determine the strategic cancellation test identification plan carried out by the patient during the identification of the plurality of symbols.

11. The method of claim 10, further comprising the step of generating a report comprising the determined strategic cancellation test identification plan carried out by the patient during the identification of the plurality of symbols.

12. The method of claim 10, wherein the step of adjusting the polar coordinates using the first factor or second factor comprises determining a first derivative comprising a difference between each identified symbol compared to its predecessor.

13. The method of claim 12, wherein the step of adjusting the polar coordinates comprises adjusting a polar coordinate by a first factor when the first derivative exceeds π, the first factor comprising subtracting 2π from the polar coordinate, and further comprises adjusting a polar coordinate by a second factor when the first derivative exceeds −π, the second factor comprising adding 2π to the polar coordinate.

14. The method of claim 12, wherein the step of adjusting the polar coordinates comprises adjusting a polar coordinate by one or more degrees.

15. The method of claim 10, wherein the step of analyzing the resulting adjusted polar coordinates comprises comparing the adjusted polar coordinates to unadjusted polar coordinates of a reference signal.
